(19) United States
(12) Plant Patent Application Publication
(10) Pub. No.: US 2018/0139878 P1
(43) Pub. Date: May 17, 2018

(54) Hop plant named 'HBC 638'

(50) ;

(71) Applicant: HOP BREEDING COMPANY, L.L.C., Yakima, WA (US)

(72) Inventors: Eugene G. Probasco, Yakima, WA (US); Jason Perrault, Toppenish, WA (US)

(21) Appl. No.: 15/732,376

(22) Filed: Oct. 31, 2017

Related U.S. Application Data
(60) Provisional application No. 62/496,943, filed on Nov. 1, 2016.

(51) Int. Cl. A01H 5/02

(52) U.S. Cl. PLT/236

(57) ABSTRACT

A new hop plant named ‘HBC 638’ is disclosed. ‘HBC 638’ is used for its exceptional and unique aromatic qualities.

### CROSS-REFERENCE TO RELATED APPLICATIONS

Priority is claimed with respect to U.S. Provisional Patent Application 62/496,943 filed on Nov. 1, 2016.

### STATEMENT REGARDING FEDERALLY SPONSORED RESEARCH OR DEVELOPMENT

None.

Genus and species: Humulus lupulus.

### Variety denomination: ‘HBC 638’.

### BACKGROUND OF THE INVENTION

‘HBC 638’ is a product of a controlled breeding program carried out by the inventors in the Yakima Valley of Washington State. ‘HBC 638’ was one of several seedlings resulting from a controlled cross made in 2008 with female parent HBC 366 (unpatented) and male parent 21-10-21. Seedling plants from this cross were planted in 2009 and screened for disease resistance and sex in a greenhouse and field nursery near Granger, Wash. A single plant of ‘HBC 638’ was selected in 2011. In 2012 ‘HBC 638’ was expanded by asexual tissue culture propagation to 14 plants in an evaluation block near Toppenish, Wash. In 2014 ‘HBC 638’ was further expanded by asexual tissue culture propagation to a 1 acre test block near Toppenish, Wash. The ‘HBC 638’ plants have now been observed and evaluated for several years. Throughout several generations of asexual propagation, ‘HBC 638’ has been observed to retain its distinctive characteristics and remain true to type.

### BRIEF DESCRIPTION OF THE PHOTOGRAPHS

FIG. 1 illustrates a mature ‘HBC 638’ hop plant grown on a trellis;

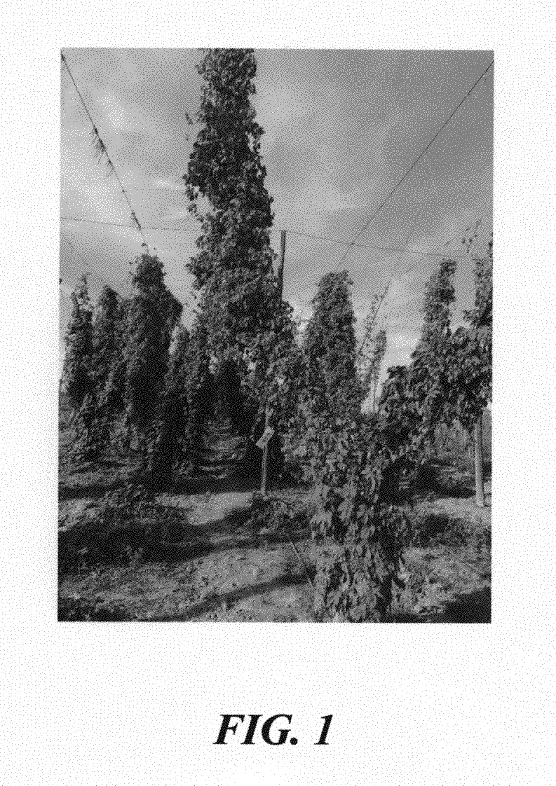

FIG. 2 illustrates whole cones and cross sections of cones of the ‘HBC 638’ hop plant;

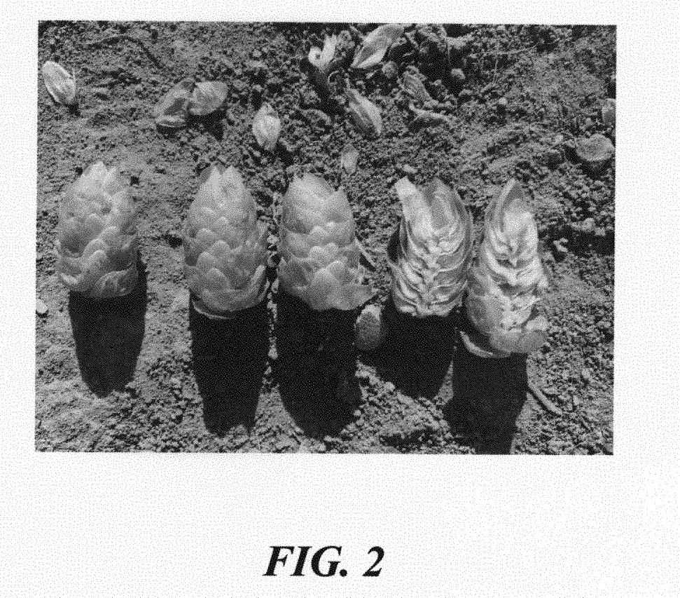

FIG. 3 illustrates the bine of a mature ‘HBC 638’ hop plant; and.

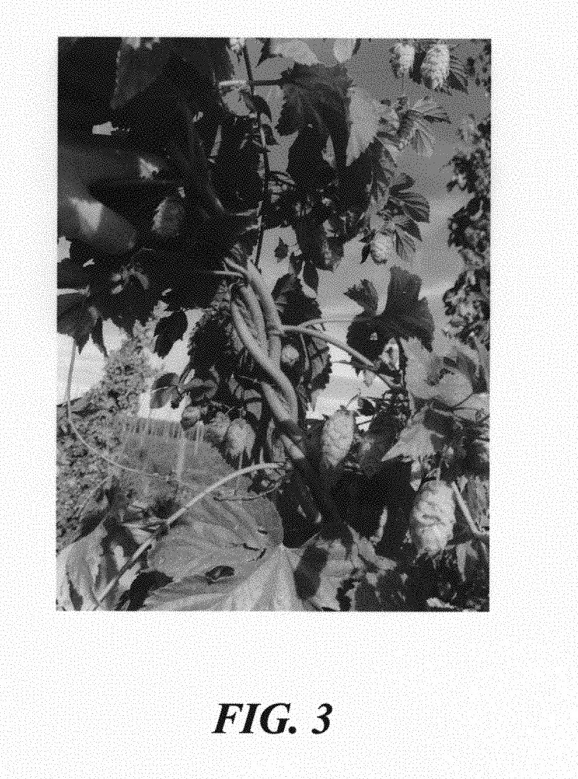

FIG. 4 illustrates cones on a mature ‘HBC 638’ hop plant.

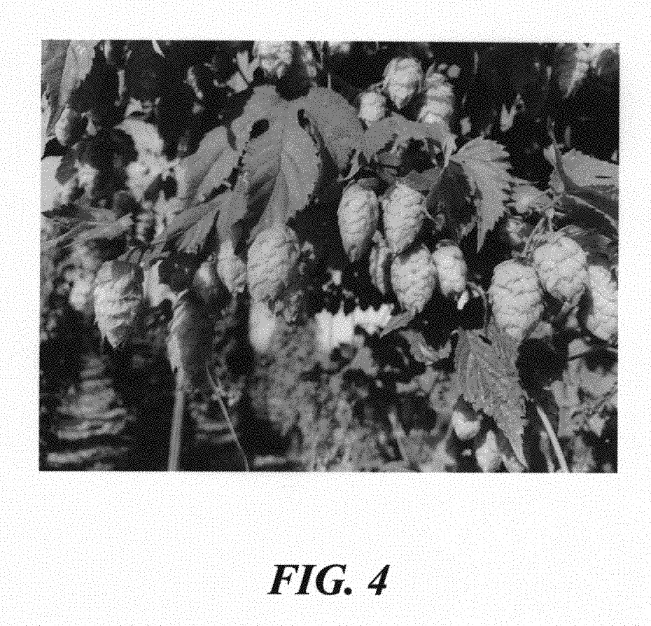

FIG. 5 illustrates leaves on a mature ‘HBC 638’ hop plant.

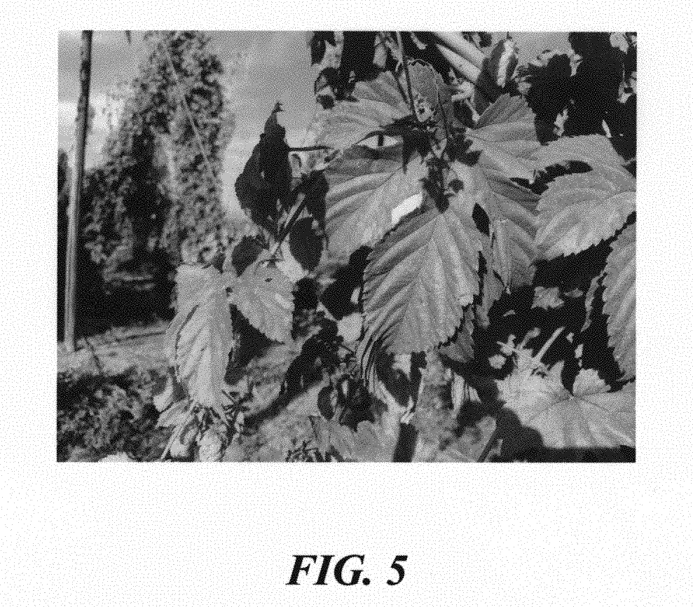

FIG. 6 illustrates an additional view of leaves on a mature ‘HBC 638’ hop plant.

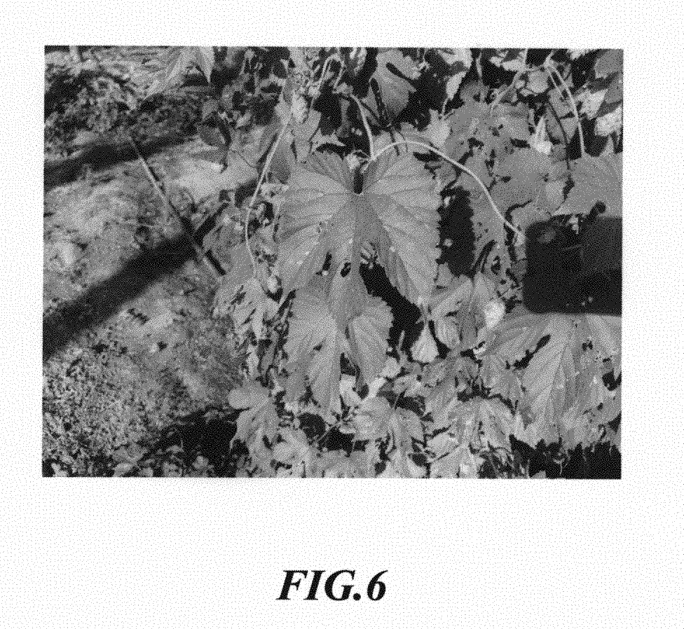

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2016 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid (2n=2x).
Sex: Female.
Use: Brewing beer and ale.
Harvest date: September 20-25 (during 2014 to 2016 growing seasons at Toppenish, Wash.).
Yield: Average yield is 2020 to 2450 kg/hectare.
Disease reaction:
   Powdery mildew.—Resistant.
   Downy mildew.—Unknown.
   Verticillium wilt.—Unknown.
Oil analytical characteristics (Averages):

| | |
|---|---|
| % Alpha Acid (% cone weight) | 13.5 to 14.5% |
| % Beta Acid (% cone weight) | 3.5 to 5.5% |
| % Cohumulone (% alpha | 28.5 to 30.5% |
| acids) | |
| Total Oil (ml/100 g of cones): | 2.30 to 3.30% Oil |
| Hop Storage Index | <20% |
| Aroma Profile | Citrus (lemon-lime), tropical, |
| | floral, pine, cedar |
| A-Pinene: | 0.07% Total Oil |
| B-Pinene: | 0.48% Total Oil |
| Myrcene: | 37.58% Total Oil |
| 2-methyl-butyl isobutyrate: | 1.48% Total Oil |
| Limonene: | 0.16% Total Oil |
| Linalool: | 0.31% Total Oil |
| Caryophyllene: | 11.42% Total Oil |
| Farnesene: | 0.12% Total Oil |
| Humulene: | 17.76% Total Oil |
| Citral: | 1.27% Total Oil |
| Geranyl Acetate: | 0.06% Total Oil |
| Citronellol: | 3.60% Total Oil |
| Nerol: | 0.39% Total Oil |
| Geraniol: | 0.19% Total Oil |
| B-lonene: | 0.01% Total Oil |

Bine:
   Color.—Yellow-Green 145B.
   Stripe present.—Yes.
   Stripe color.—Red-Purple 60B.
   Stipule direction.—Up.
   Bine diameter.—9.4 mm at base; 9.6 mm at nine feet; 7.8 mm at terminal end of eighteen feet.
Leaf:
   Arrangement.—Opposite.
   Shape.—Palmate.
   Average length of mature leaf.—13.7 cm.
   Average width of mature leaf.—15.3 cm.
   Color of mature leaf upper surface.—Yellow-Green 147A.
   Color of mature leaf lower surface.—Yellow-Green 147B.
   Color of immature leaf upper surface.—Yellow-Green 146B.
   Color of immature leaf lower surface.—Yellow-Green 147B.
   Number of lobes.—1, 3 or 5.
   Margin.—Serrate.
   Serrations per inch.—5-7.
   Average petiole length (mature).—81 mm.
   Petiole color at base.—Yellow-Green 144B.
   Venation.—Palmate.
   Vein color.—Yellow-Green 144B.
Cone:
   Avg. Length: 31 mm.
   Avg. diameter: 15 mm.
   Bract tip color.—Yellow-Green 144A.
   Bract base color.—Yellow-Green 145A.
   Bracteole color.—Yellow-Green 145B.
   Cone shape.—Ovate.
   Bract shape.—Ovate.
   Bract tip shape.—Acuminate-Mucronate.
   Bract tip position.—Recurved.
   Bracteole shape.—Oval.
   Pickability.—Good.
   Lupulin glands color.—Yellow 4A.

1. A new and distinct Hop Plant as illustrated and described herein.
